(19) United States
(12) Patent Application Publication
(10) Pub. No.: US 2004/0043940 A1
(43) Pub. Date: Mar. 4, 2004

(54) Use of ectoin or ectoin derivatives for protecting stress proteins in the skin

(76) Inventors: Joachim Bünger, (US); Francois Marchio, Scarsdale, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/239,394

(22) PCT Filed: Mar. 15, 2001
(86) PCT No.: PCT/EP01/02987

(30) Foreign Application Priority Data
Mar. 24, 2000 (DE) 100 14 632.5

(51) Int. Cl. A61K 38/06; A61K 38/05; A61K 31/551; A61K 31/505; A61K 31/4172

(52) U.S. Cl. 514/18; 514/19; 514/218; 514/256; 514/399

(57) ABSTRACT

The present invention relates to the use of at least one compound selected from the group comprising compounds of formulas 1a and 1b

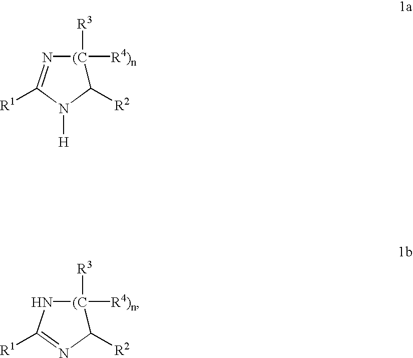

physiologically acceptable salts thereof, and stereoisomeric forms thereof, in which

R¹ denotes H oder alkyl,

R² denotes H, COOH, COO-alkyl or CO—NH—R⁵,

R³ and R⁴ each independently denote H or OH,

n is 1, 2 or 3,

R⁵ denotes H, alkyl, an amino acid group, a dipeptide residue or a tripeptide residue, and

alkyl denotes an alky group containing from 1 to 4 carbons,

for protection of stress proteins in the skin.

These compounds are used in the present invention in the form of a topical composition.

The present invention relates to the use of ectoin or ectoin derivatives for protection of stress proteins in the skin.

The skin, as the boundary surface of the human body, is exposed to a large number of environmental stress factors. The human skin is an organ which protects the body from external influences by means of a variety of specialized cell types, such as the keratinocytes, the melanocytes, Langerhans cells, Merkel's cells, and embedded sense cells. These external influences on the human skin should be distinguished according to whether they are of a physical, chemical, or biological nature. The physical external influences include thermal and mechanical effects and also the action of radiation, such as UV and IR radiation. By chemical external influences are meant, in particular, the action of toxins and allergens. Biological external influences comprise the action of foreign organisms and their metabolic products. Further stress factors are pathological states and diseases, such as pyrexia, inflammation, infection, cell and tissue trauma, and also physiological processes, such as cytokinesis.

The synthesis of stress proteins is an important part of the cellular response to these different stress factors. These stress proteins have a protective action and counteract damaging influences on the cells. They are therefore also known as molecular chaperones.

The group of constitutive stress proteins is also expressed under unstressed, or normal, conditions and also during developmental and differential processes. The presence thereof is essential for correct folding, assembly, stabilization, transfer, and degradation of other proteins. In addition, the cell possesses a spectrum of inducible stress proteins which are synthesized in response to stress in order to counterbalance damage caused thereby.

The stress proteins were first discovered in Drosophila melanogaster. They were caused by a heat shock, which gave rise to the expression heat-shock proteins (HSP). Subsequent research showed that stress proteins are detectable in every cell: in the living organism, ie from the bacterium through plants to the mammal, in organ transplants, and in cell cultures (A. Hubel (1992) Die Hitzeschockantwort, Biologie in unserer Zeit 3: 281-285). It is found that stress proteins have been phylogenetically highly conserved during evolution. Thus the HSPs of bacteria differ from those of mammals to a relatively minor degree.

Each cell possesses an intrinsic quantitatively and, in some cases, qualitatively typical repertoire of constitutive and inducible stress proteins. Alteration of this repertoire after exposure to stress is called stress response and differs specifically for each type of cell and for each type of stress. Thus a temperature of 42° C. is regarded as a heat shock to human fibroblasts and is associated with a stress response (M. J. Edwards, R. Marks, P. Dyks, V. Merrett, H. Morgan & M. O'Donovan (1991) Heat Shock Proteins in Cultured Human Keratinocytes and Fibroblasts—The J. Invest. Dermat. 96: 392-395; H. Marshall & C. Kind (1994) Detection and Cellular Localization of Stress-induced 72 kD Heat Shock Protein in Cultured Swiss 3T3 Mouse Fibroblasts—Toxic. in vitro 8: 545-548) while in the case of hepatocytes only higher temperatures than this initiate a stress response. In the same way, the stress responses of identical cell types of different organisms differ depending on their optimum temperature (S. Lndquist (1986) The heat-shock response—Ann. Rev. Biochem. 55: 1151-1191). The stress responses to various stress factors likewise differ. Despite many parallels as regards composition and quantity of the expressed stress proteins, the heat-shock response differs from the cold-shock response (P Jones. & M. Inouye (1994) The cold-shock response—a hot topic—Mol. Microbiol. 11: 811-818). Evidence of variance from the UV stress response has also been found (T. Muramatsu, H. Tada, N. Kobayashi, M. Yamaji, T. Shirai & T. Ohnishi (1992) Induction of the 72 kD heat shock protein in organ-cultured normal human skin—J. Invest. Dermat. 98: 786-790). The stress proteins HSP 60, HSP 90, and HSP 72/73 have been found and studied.

HSP 60 belongs to the class of constitutive stress proteins. In prokaryota it is referred to as “groEL” and is regarded as being essential for the growth of bacteria, in which it represents, in the unstressed state, approximately 1.5% of the total amount of protein.

In eukaryotes, HSP 60 has been located in the mitochondrial template (in Tetrahymena, yeast, Xenopus and human cells) (T. Langer, C. Lu, H. Echols, J. Flanagan, M. Hayer & F. Hartl (1992) Successive action of DnaK, DnaJ and GroEL along the pathway of chaperone mediated protein folding—Nature 356: 683-689). One function is the mediation of correct folding and assembly of proteins imported from the cytosol within the mitochondria. This is performed by HSP 60 not only on proteins which are normally assigned to the mitochondrial template, such as the β sub unit of F₁-ATPase, but also on those having complex presequences, such as cytochrome b₂, whose destination lies in the mitochondrial intermembranous space (M. Gething & J. Sambrook (1992) Protein folding in the cell—Nature 355: 33-45). The latter, following cleavage of their presequences, are converted to a form suitable for traversing the inner mitochondrial membrane. Correct folding of a functional HSP 60 from the monomeric “transportable form” to a reactive, decatetrameric form is self-mediated (D. Ang., K. Liberek, K. Skowyra, M. Zylicz & C. Georgopoulos (1991) Biological Role and Regulation of the Universally Conserved Heat Shock Proteins—J. Biol. Chem. 266: 24233-24236).

The stress protein HSP 90 also belongs to the group of constitutive HSPs. It is thus also expressed in the unstressed state (L. Hightower (1991) Heat shock, Stress proteins, Chaperones and Proteotoxicity—Cell 66: 191-197). Two different versions of the HSP 90 gene have been located, a constitutively-expressing version and an inducible version (D. Ang., K. Liberek, K. Skowyra, M. Zylicz & C. Georgopoulos (1991) Biological Role and Regulation of the Universally Conserved Heat Shock Proteins—J. Biol. Chem. 266: 24233-24236).

HSP 90 is associated with various steroid hormone receptors in the cytosol of the cell in a complex with HSP 70 and HSP 56 (E. Sanchez, L. Faber, W. Henzel & W. Pratt (1990) The 56-59-Kilodalton Protein Identified in Untransformed Steroid Receptor Complexes Is a Unique Protein That Exists in Cytosol in a Complex with both the 70- and 90-kilodalton Heat Shock Proteins—Biochemistry 29: 5145-5152; M. Czar, J. Owens-Grillo, K. Dittmar, K. Hutchinson, A. Zacharek, K. Leach, M. Deibel & W. Pratt (1994) Characterization of the Protein-Protein Interactions Determining the Heat Shock Protein (hsp90.hsp70.hsp56) Heterocomplex. J. Biol. Chem. 269: 11155-11161), in order to stabilize it in an inactive form (H. Wiech, J. Buchner, R. Zimmermann & U. Jakob (1992) HSP 90 chaperones protein folding in vitro—Nature 358: 169-170). If steroid hormones (progesterone, oestrogen, and glucocorticoids) occur, they liberate HSP 90, bind to the steroid hormone receptor and can then interact with the DNA as an activated receptor complex in the cell nucleus, to activate transcription (A. Baniahmad & M.-J. Tsai (1993) Mechanisms of Transcriptional Activation by Steroid Hormone Receptors—J. Cell. Biochem. 51: 151-156; D. Smith, L. Faber & D. Toft (1990) Purification of Unactivated Progesterone Receptor and Identification of Novel Receptor-associated Proteins—J. Biol. Chem. 265: 3996-4003). The formation of HSP 90 on the steroid hormone receptor is regarded as a prerequisite for its assembly with the steroid hormone (U. Jakob & J. Buchner (1994) Assisting spontaneity: the role of Hsp 90 and small Hsps as molecular chaperones—Trends Biochem. Sci. 19: 205-211).

HSP 72 and 73 are regarded as cytoplasmatically-occuring versions of the so-called “HSP 70 family” (R. Beckmann, L. Mizzen & W. Welch (1990) Interaction of Hsp70 with Newly Synthesized Proteins: Implications for Protein Folding and Assembly—Science 248: 850-854). Members of this HSP 70 family have been detected in prokaryotes, yeast, and higher eukaryotes. The sole representative in Escherischia coli is the so-called DnaK, whilst in eukaryotes there exist several inducible and constitutive forms (D. Palleros, L. Shi, K. Reid & A. Fink (1994) hsp70-Protein Complexes—J. Biol. Chem. 269: 13107-13114). They have also been localized in the cell in nucleoplasms, in plastids, in mitochondria, and in the endoplasmic reticulum (S. Rensing & U. Maier (1994) Phylogenetic analysis of the stress-70 protein family—J. Mol Evolut. 39: 80-86). Their function may be seen to be the mediation of correct folding of proteins which have been freshly synthesized or damaged, and the part they play in the transference of proteins through membranes. This task is achieved by these HSPs with consumption of ATP (G. Flynn, T. Chappell & J. Rothman (1989) Peptide Binding and Release by Proteins Implicated as Catalysts of Protein Assembly—Science 245: 385-390), which is required in order to release the bond between HSP and the substrate (D. Palleros, L. Shi, K. Reid, W. Welch & A. Fink (1993) ATP-induced protein-Hsp70 complex dissociation requires K⁺ but not ATP hydrolysis—Nature 365: 664-666).

For protection of the cells it is always important to have an adequate concentration of stress proteins to defend the cells against the various stress factors. However, the existing concentration of stress proteins may be reduced by the action of various stress factors. This weakens the defence status of the cells so that there is inadequate reaction to the different stress factors. In other words, the stress effect can result in a condition in which the constitutive concentration of stress proteins is too low and/or the synthesis of stress protein takes place at an inadequate rate.

It is thus an object of the present invention to overcome, or at least alleviate, the aforementioned problems and to provide a compound which protects the stress proteins in the skin such that they remain present in adequate concentration, in order to improve the defence status of skin cells.

This object is achieved by the use of at least one compound selected from the group comprising compounds of formulas 1a and 1b:

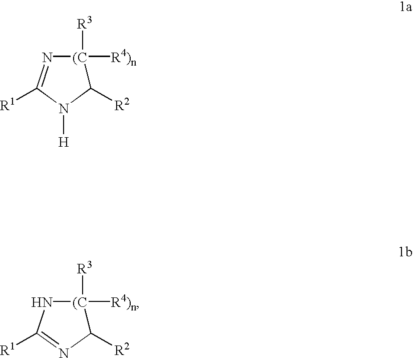

physioligically acceptable salts thereof, and stereoisomeric forms thereof, in which

R¹ denotes H oder alkyl,

R² denotes H, COOH, COO-alkyl or CO—NH—R⁵,

R³ und R⁴ each independently denote H or OH,

n is 1, 2 or 3,

R⁵ denotes H, alkyl, an amino acid group, a dipeptide residue or a tripeptide residue, and

alkyl denotes an alkyl group containing from 1 to 4 carbons,

for the protection of the stress proteins in the skin.

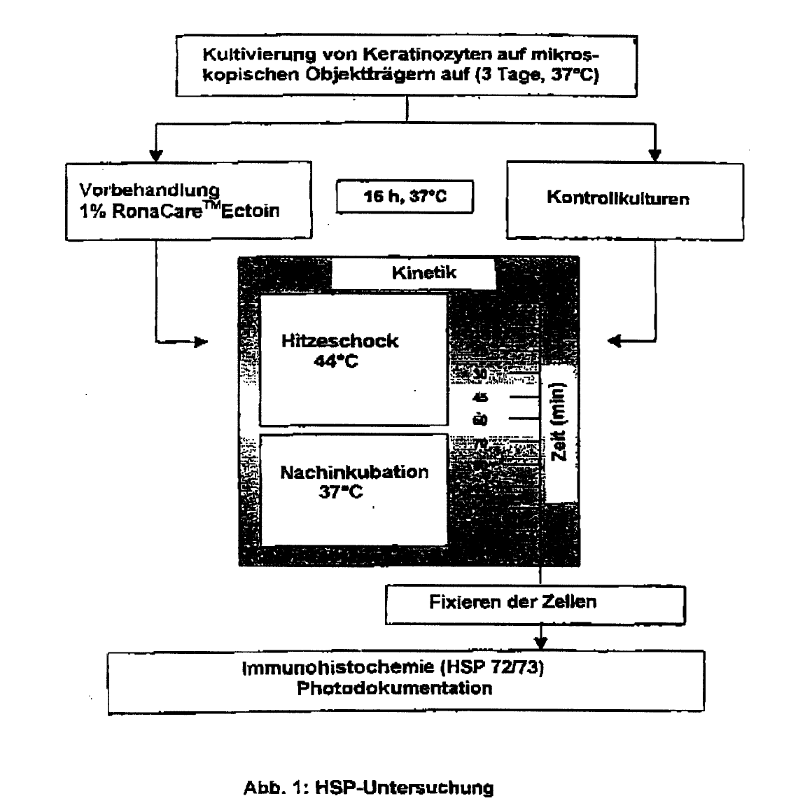

FIG. 1 presents a summary of tests on the induction of stress proteins, as described in Example 30.

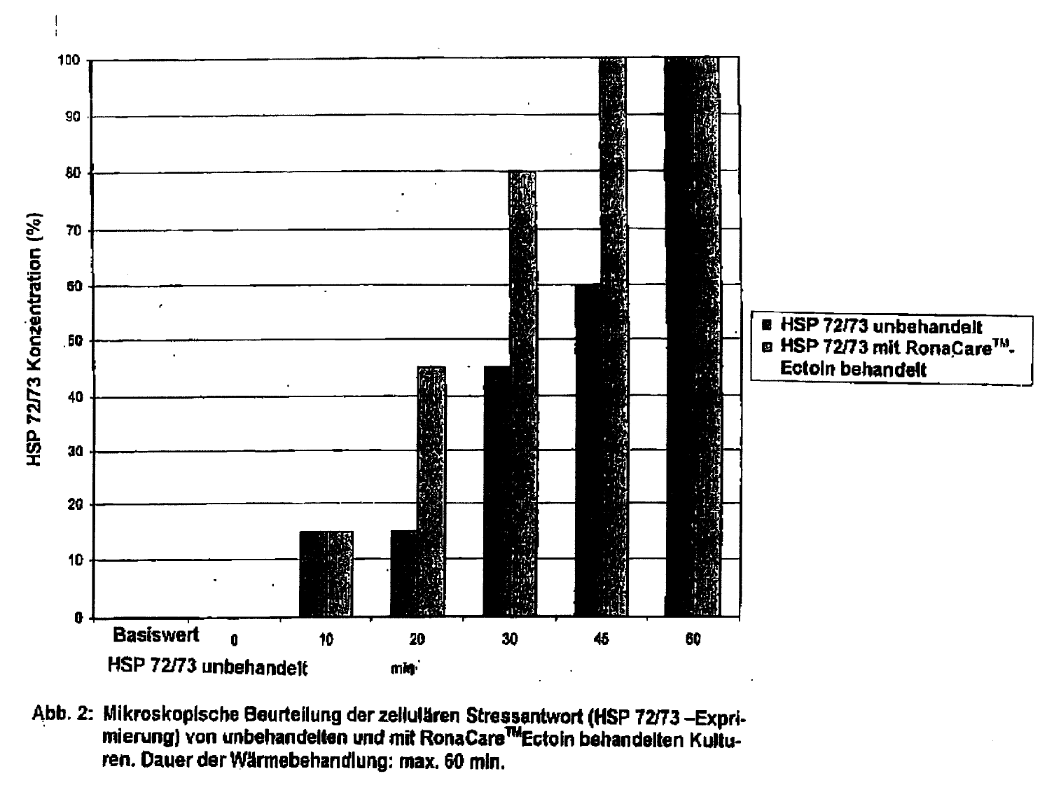

FIG. 2 shows the microscopic assessment of the cellular stress response as HSP 72/73 concentration over a period of 60 minutes, as provided by the HSP experiments carried out in Example 30.

The compounds of formulas 1a and 1b, the physiologically acceptable salts of the compounds of formulas 1a and 1b, and the stereoisomeric forms of the compounds of formulas 1a and 1b are referred to below as “ectoin or ectoin derivatives”.

Ectoin and the ectoin derivatives are low-molecular, cyclic amino-acid derivatives obtained from various halophilic microorganisms. Both ectoin and ectoin derivatives have the advantage that they do not interfere with cell metabolism. Ectoin and ectoin derivatives have already been described in DE 43 42 560 as moisturizing agents for use in cosmetic products.

The compounds used in the present invention can be present in topical formulations in the form of optical isomers, diastereoisomers, racemates, zwitterions, cations, or a mixture thereof.

The compounds used in the present invention are preferably those in which R¹ denotes H or CH₃, R² denotes H or COOH, R³ and R⁴ independently denote H or OH, and n is 2. Of the compounds used in the present invention, (S)-1,4,5,6-tetrahydro-2-methyl-4-pyrimidinecarboxylic acid (ectoin) and (S,S)-1,4,5,6-tetrahydro-5-hydroxy-2-methyl-4-pyrimidinecarboxylic acid (hydroxylectoin) are particularly preferred.

By the term “amino acid” we mean the stereoisomeric forms, eg, D and L forms of the following compounds: alanine, β-alanine, arginine, asparagine, aspartic acid, cysteine, glutamine, glutamic acid, glycine, histidine, isoleucine, leucine, lysine, methionine, phenylalanine, serine, threonine, tryptophane, tyrosine, valine, γ-aminobutyrate, Nε-acetyllysine, Nδ-acetylornithine, Nγ-acetyidiaminobutyrate, and Nα-acetyldiaminobutyrate. L-amino acids are preferred.

Amino-acid residues are derived from the corresponding amino acids.

The residues of the following amino acids are preferred: alanine, β-alanine, asparagine, aspartic acid, glutamine, glutamic acid, glycine, serine, threonine, valine, γ-aminobutyrate, Nε-acetyllysine, Nδ-acetylornithine, Nγ-acetyldiaminobutyrate, and Nα-acetyldiaminobutyrate.

The dipeptide and tripeptide residues are acid amides by chemical nature and decompose under hydrolysis to two or three amino acids. The amino acids in the dipeptide and tripeptide residues are bonded to each other by amide linkages. Preferred dipeptide and tripeptide residues are based on the preferred amino acids.

The alkyl groups comprise the methyl group CH₃, the ethyl group C₂H₅, the propyl groups CH₂CH₂CH₃ and CH(CH₃)₂, and the butyl groups CH₂CH₂CH₂CH₃, H₃CCHCH₂CH₃, CH₂CH(CH₃)₂, and C(CH₃)₃. The preferred alkyl group is the methyl group.

Preferred physiologically acceptable salts of the compounds used in the present invention are, for example, alkali salts, alkaline earth metal salts, or ammonium salts, such as Na, K, Mg, or Ca salts, and also salts which are derived from the organic bases triethylamine or tris(2-hydroxyethyl)amine. Other preferred physiologically acceptable salts of the compounds used in the present invention are obtained by reaction with inorganic acids, such as hydrochloric acid, sulfuric acid, and phosphoric acid, or with organic carboxylic or sulfonic acids, such as acetic acid, citric acid, benzoic acid, maleic acid, fumaric acid, tartaric acid, and p-toluene-sulfonic acid.

Compounds of formulas 1a and 1b in which base groups and acid groups, such as carboxyl or amino groups, are present in equal number, form internal salts.

The production of the compounds used in the present invention is described in DE 43 42 560. (S)-1,4,5,6-tetrahydro-2-methyl-4-pyrimidinecarboxylic acid or (S,S)-1,4,5,6-tetrahydro-5-hydroxy-2-methyl-4-pyrimidinecarboxylic acid may alternatively be obtained microbiologically (Severin et al, J. Gen. Microb. 138 (1992) 1629-1638).

According to the invention, ectoin or ectoin derivatives are usually employed in the form of a topical composition.

The production of the topical composition is effected by converting at least one of the compounds used in the present invention, optionally together with adjuvents and/or vehicles, to a suitable formulation form. The adjuvants and vehicles are selected from the group comprising vehicles, preservatives, and other conventional adjuvants.

The topical composition based on at least one compound used in the present invention is applied externally to the skin or skin adnexa.

As examples of suitable administration forms there may be mentioned: solutions, suspensions, emulsions, pastes, ointments, gels, creams, lotions, powders, soaps, surfactant-containing cleaning preparations, oils, and sprays. In addition to one or more compounds used in the present invention, any conventional vehicles, adjuvants, and, optionally, other active substances may be added to the composition.

Preferred adjuvants are selected from the group comprising preservative agents, antioxidants, stabilizing agents, solutizers, vitamins, coloring agents, and odor improvers.

Ointments, pastes, creams, and gels may contain, in addition to one or more compounds used in the present invention, conventional vehicles, eg, animal and vegetable fats, waxes, paraffin waxes, starch, gum traganth, cellulose derivatives, polyethylene glycols, silicones, bentonites, silicic acid, talcum powder, zinc oxide, or mixtures of these materials.

Powders and sprays may contain, in addition to one or more compounds used in the present invention, conventional vehicles, eg, lactose, talcum powder, silicic acid, aluminium hydroxide, calcium silicate, polyamide powder, or mixtures of these materials. Sprays can additionally contain conventional aerosol propellants, eg, chlorofluorocarbons, propane/butane, or dimethyl ether.

Solutions and emulsions may contain, in addition to one or more compounds used in the present invention, conventional vehicles, such as solvents, solutizers, and emulsifiers, eg, water, ethanol, isopropanol, ethyl carbonate, ethyl acetate, benzyl alcohol, benzyl benzoate, propylene glycol, 1,3-butyl glycol, oils, particularly cottonseed oils, peanut oil, maize germ oil, olive oil, castor oil, and sesame oil, glycerin fatty acid esters, polyethylene glycols, fatty acid esters of sorbitan, or mixtures of these materials.

Suspensions may contain, in addition to one or more compounds used in the present invention, conventional vehicles, such as liquid diluents, eg, water, ethanol, or propylene glycol, suspending agents, eg, ethoxylated isostearyl alcohols, polyoxyethylene sorbitol ester, and polyoxyethylene sorbitan ester, microcrystalline cellulose, aluminium metahydroxide, bentonite, agar-agar, gum traganth, or mixtures of these materials.

Soaps may contain, in addition to one or more compounds used in the present invention, conventional vehicles, such as alkali-metal salts of fatty acids, salts of fatty acid half-esters, fatty acid albumin hydrolysates, isothionates, lanoline, fatty alcohol, plant oils, plant extracts, glycerin, sugar, or mixtures of these materials.

Surfactant-containing cleaning products may contain, in addition to one or more compounds used in the present invention, conventional vehicles, such as salts of fatty alkyl sulphates, fatty alcohol ether sulfates, sulfosuccinic acid half-esters, fatty acid albumin hydrolysates, isothionates, imidazolinium derivatives, methyl taurates, sarcosinates, fatty amide ether sulfates, alkyl amidobetaines, fatty alcohols, fatty acid glycerides, fatty acid diethanolamides, vegetable and synthetic oils, lanoline derivatives, ethoxylated glycerin fatty acid esters, or mixtures of these materials.

Face oils and body oils may contain, in addition to one or more compounds used in the present invention, the usual vehicles, such as synthetic oils, for example fatty acid esters, fatty alcohols, silicone oils, and natural oils, such as plant oils and oily plant extracts, paraffin oils, lanoline oils, or mixtures of these materials.

Other, typically cosmetic, administration forms are lipsticks, lip-care sticks, mascara, eyeliners, eye-shadow, rouge, powder make-up, emulsion make-up, wax make-up, sun-screening preparations, and pre-sun preparations and after-sun preparations.

At least one compound used in the present invention is present in the topical composition in a concentration of preferably from 0.0001 to 50 wt %, more preferably from 0.001 to 10 wt %, and most preferably from 0.1 to 1 wt %, based on the composition.

Preferably, at least one antioxidant and/or UV filter is used in addition to ectoin or the ectoin derivatives.

According to the invention, the antioxidants disclosed in the technical literature may be used, for example, flavonoids, coumaranones, amino acids (eg, glycine, histidine, tyrosine, tryptophane) and derivatives thereof, imidazoles, (eg, urocanic acid) and derivatives thereof, peptides, such as D,L-carnosine, D-carnosine, L-carnosine, and derivatives thereof (eg, anserine), carotenoids, carotenes (eg, α-carotene, β-carotene, lycopene) and derivatives thereof, chlorogenic acid and derivatives thereof, lipoic acid and derivatives thereof (eg, dihydrolipoic acid), aurous thioglucose, propyl thiouracil and other thiols (eg, thioredoxin, glutathione, cysteine, cystine, cystamine and their glycosyl, N-acetyl, methyl, ethyl, propyl, amyl, butyl, and lauryl, palmitoyl, oleyl, γ-linoleyl, cholesteryl, and glycerin esters) and also their salts, dilauryl thiodipropionate, distearyl thiodipropionate, thiodipropionic acid and derivatives thereof (esters, ethers, peptides, lipids, nucleotides, nucleosides, and salts) and also sulfoximine compounds (eg, buthioninsulfoximines, homocysteinesulfoximine, buthioninsulfones, penta-, hexa-, or hepta-thioninesulfoximine), further (metallic) chelating agents (eg, α-hydroxyl fatty acids, palmitic acid, phytic acid, lactoferrin), α-hydroxyl acid (eg, citric acid, lactic acid, malic acid), humic acid, bile acid, biliary extracts, bilirubin, biliverdin, EDTA, EGTA and derivatives thereof, unsaturated fatty acids and derivatives thereof, vitamin C and derivatives (eg, ascorbyl palmitate, magnesium ascorbyl phosphate, ascorbyl acetate) and also coniferyl benzoate of benzoin, rutinic acid and derivatives thereof, α-glycosyl rutin, ferulic acid, furfurglucitol, carnosine, butyl hydroxyltoluene (BHT), butylated hydroxyanisole, nordohydroguaiaretic acid, and trihydroxybutyrophenone, uric acid and derivatives thereof, mannose and derivatives thereof, zinc and derivatives thereof (eg, ZnO, ZnSO₄), selenium and derivatives thereof (eg, selenomethionine), and stilbenes and derivatives thereof (eg, stilbene oxide and trans-stilbene oxide).

Mixtures of antioxidants are equally suitable. Known and commercial mixtures are, for example, mixtures containing as active constituents lecithin, L-(+)-ascorbyl palmitate, and citric acid (eg, Oxynex® AP), natural tocopherols, L-(+)-ascorbyl palmitate, L-(+)-ascorbic acid, and citric acid (eg, Oxynex® K LIQUID), tocopherol extracts from natural sources, L-(+)-ascorbyl palmitate, L-(+)-ascorbic acid, and citric acid (eg, Oxynex® L LIQUID), D,L-α-tocopherol, L-(+)-ascorbyl palmitate, citric acid and lecithin (eg, Oxynex® LM) or butyl hydroxytoluene (BHT), L-(+)-ascorbyl palmitate, and citric acid (eg, Oxynex® 2004).

In a preferred embodiment of the invention, the antioxidant used is butyl hydroxytoluene. In another preferred embodiment, the antioxidant used comprises one or more compounds selected from the group comprising flavonoids and/or coumaranones.

Flavanoids are taken to mean glycosides of flavanones, flavones, 3-hydroxy-flavones (=flavanols), aurones, isoflavones, and rotenoids (Rompp Chemie Lexikon, Vol. 9, 1993). However, for the purposes of the present invention, they are also taken to include the aglycons, ie aglycosuric components, and the derivatives of said flavonoids and aglycons. For the purposes of the present invention, coumaranones are also taken to include their derivatives.

Preferred flavonoids are derived from flavanones, flavones, 3-hydroxyflavones, aurones, and isoflavones, particularly from flavanones, flavones, 3-hydroxyflavones, and aurones.

The flavanones are characterized by the following basic structure:

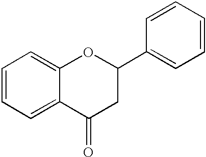

The flavones are characterized by the following basic structure:

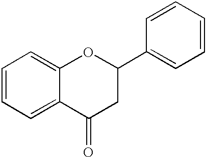

The 3-hydsroxyflavones (flaonols) are characterized by the following basic structure:

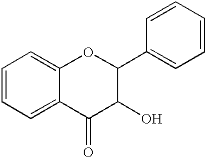

The isoflavones are characterized by the following basic structure:

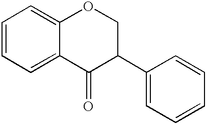

The aurones are characterized by the following basic structure:

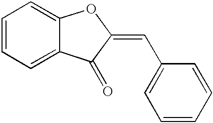

The coumaranones are characterized by the following basic structure:

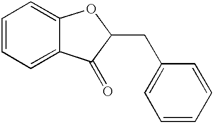

Preferably, the flavonoids and coumaranones are selected from the group comprising the compounds of formula (I):

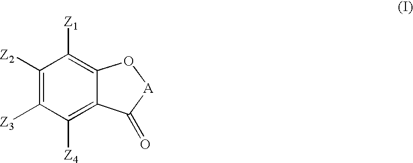

in which

Z₁ to Z₄ independently denote H, OH, alkoxy, hydroxyalkoxy, and mono- or oligo-glycoside groups, in which the alkoxy and hydroxyalkoxy groups can be branched or unbranched and can exhibit from 1 to 18 carbons and in which sulfate or phosphate may also be bonded to the hydroxyl groups in said groups,

A is selected from the group comprising the partial forms (IA), (IB) and (IC)

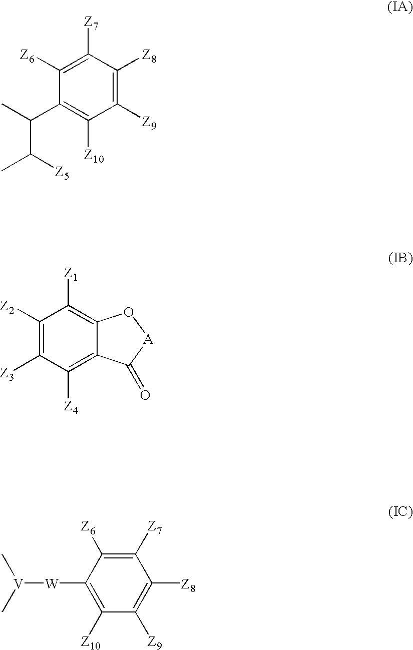

in which

Z₅ denotes H, OH, or OR,

R denotes a monoglycoside or oligoglycoside group,

Z₆ to Z₁₀ have the meanings given above for Z₁ to Z₄, or denote

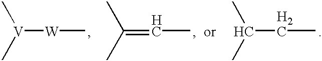

The alkoxyl groups are preferably linear and possess from 1 to 12 and preferably from 1 to 8 carbons. Thus these groups conform to the formula —O—(CH₂)ₘ—H, in which m is 1, 2, 3, 4, 5, 6, 7, or 8, and, in particular, 1 to 5.

The hydroxyalkoxy groups are preferably linear and possess from 2 to 12, and preferably from 2 to 8 carbon atoms. Thus these groups conform to the formula —O—(CH₂)ₙ—OH, in which n is 2, 3, 4, 5, 6, 7, or 8, preferably from 2 to 5, and more preferably 2.

The monoglycoside and oligoglycoside groups are preferably composed of from 1 to 3 glycoside units. Preferably, these units are selected from the group comprising hexosyl residues, and particularly rhamnosyl residues and glucosyl residues. Alternatively, other hexosyl residues, for example, allosyl, altrosyl, galactosyl, gulosyl, idosyl, mannosyl, and talosyl, may be used to advantage, if desired. Alternatively, it may be advantageous to use pentosyl residues in the present invention.

In a preferred embodiment, the meanings of the variants are as follows:

Z₁ and Z₃ each denote H,

Z₂ and Z₄ are other than H, and denote, in particular, OH, methoxy, ethoxy or 2-hydroxyethoxy,

Z₅ denotes H, OH, or a glycoside residue composed of from 1 to 3, and preferably 1 or 2, glycoside units,

Z₆, Z₉ and Z₁₀each denote H, and

Z₇ and Z8 are other than H, and preferably denote OH, methoxy, ethoxy, or 2-hydroxyethoxy

In another preferred embodiment, particularly when the water solubility of the flavonoids and coumaranones is to be raised, a sulfate or phosphate group is bonded to the hydroxyl group. Suitable counterions are, for example, the ions of the alkali metals or alkaline-earth metals, these being selected from the group comprising, for example, sodium and potassium.

In another preferred embodiment, the flavonoids are selected from the group comprising the following compounds: 4,6,3′,4′-tetrahydroxyaurone, quercetin, rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), and trishydroxyethylluteolin (troxeluteolin), and their sulfates and phosphates.

Of the flavonoids, rutin and troxerutin are especially preferred, and particular preference is given to troxerutin.

Among the coumaranones, preference is given to 4,6,3′,4′-tetrahydroxybenzyl-coumaranone-3.

According to the invention, the antioxidants are used in the topical composition in conventional concentrations.

Furthermore, UV filters disclosed in the technical literature can be used in the present invention.

Suitable organic UV filters are all UVa and UVb filters known to the person skilled in the Art. For both UV ranges there are many substances which have been disclosed in the technical literature and which have been used with success, eg,

benzylidenecamphor derivatives, such as

3-(4′-methylbenzylidene)-dl-camphor (eg, Eusolex® 6300),

3-benzylidenecamphor (eg, Mexoryl® SD),

polymers of N-{(2 and 4)-[(-3-oxoborn-4-ylidene)methyl]benzyl}acrylamide (eg, Mexoryl® SW),

N,N,N-trimethyl-4-(2-oxoborn-3-ylidenemethyl)anilinium methylsulfate (eg, Mexoryl® SK) or

α-(2-oxobornyl-3-ylidene)toluene-4-sulfonic acid (eg, Mexoryl® SL),

benzoyl- or dibenzoyl-methanes, such as

1-(4-tert-butylphenyl)-3-(4-methoxyphenyl)propane-1,3-dione (eg, Eusolex® 9020) or

4-isopropyldibenzoylmethane (eg, Eusolex® 8020),

benzophenones, such as

2-hydroxy-4-methoxybenzophenone (eg, Eusolex® 4360) or

2-hydroxy-4-methoxybenzophenone-5-sulfonic acid and the sodium salt thereof (eg, Uvinul® MS 40),

methoxycinnamates; such as

2-ethylhexyl p-methoxycinnamate (eg, Eusolex® 2292),

isopentyl p-methoxycinnamate, eg, as a mixture of the isomers (eg, Neo Heliopan® E 1000),

salicylate derivatives, such as

2-ethylhexyl salicylate (eg, Eusolex® OS),

4-isopropylbenzyl salicylate (eg, Megasol®) or

13,3,5-trimethylcyclohexyl salicylate (eg, Eusolex® HMS),

4-aminobenzoic acid and derivatives thereof, such as

4-aminobenzoic acid,

2-ethylhexyl 4-(dimethylamino)benzoate (eg, Eusolex® 6007), ethoxylated ethyl 4-aminobenzoate (eg, Uvinul® P25),

and other substances, such as

2-ethylhexyl 2-cyano-3,3-diphenylacrylate (eg, Eusolex® OCR),

2-phenylbenzimidazol-5-sulfonic acid, and the potassium, sodium, and triethanolamine salts thereof (eg, Eusolex® 232)

3,3′-(1,4-phenylenedimethylene)-bis(7,7-dimethyl-2-oxobicyclo[2.2.1]heptyl-1-methanesulfonic acid, and the salts thereof (eg, Mexoryl® SX), and

2,4,6-trianilino-(p-carbo-2′-ethylhexyl-1′-oxo)-1,3,5-triazine (eg, Uvinul® T 150).

These organic UV filters are usually employed in the topical composition used in the present invention in a concentration of from 0.5 to 10 wt %, and preferably from 1 to 8 wt %.

Other suitable organic UV filters are, for example,

2-(2H-benzotriazol-2-yl)-4methyl-6-(2-methyl-3-(1,3,3,3-tetramethyl-1-(trimethylsilyloxy)disiloxanyl)propyl)phenol (eg, Silatrizole®),

4,4′-[(6-[4-((1,1-dimethylethyl)aminocarbonyl)phenylamino]-1,3,5-triazin-2,4-diyl)diimino]-bis(2-ethylhexyl benzoate) (eg, Uvasorb® HEB),

α-(trimethylsilyl)-ω-[trimethylsilyl)oxy]poly[oxy(dimethyl][and ca 6% of methyl[2-[p-[2,2-bis(ethoxycarbonyl]vinyl]phenoxy]-1-methylenethyl] and ca 1.5% of methyl[3-[p-[2,2-bis(ethoxycarbonyl)vinyl)phenoxy)propenyl) and from 0.1 to 0.4% of (methylhydrogen]silylene]] (n≈60) (eg, Parsol® SLX,

2,2′-methylene-bis(6-(2-H-benzotriazolyl-2)-4-(1,1,3,3-tetramethylbutyl)phenol (eg, Tinosorb® M),

2,2′-(1,4-phenylene)-bis(1H-benzimidazol-4,6-disulfonic acid and the monosodium salt thereof,

2,2′-(1,4-phenylene)-bis(1H-benzimidazol-5-sulfonic acid and the monosodium salt thereof,

2,2′-(1,4-phenylene)-bis(1H-benzimidazol-5-sulfonic acid and the monopotassium salt thereof, and

2,4-bis{[4-(2-ethylhexyloxy)-2-hydroxyl]phenyl}-6-(4-methoxyphenyl)-1,3,5-triazine (eg, Tinosorb® S).

These organic filters are usually employed in the topical composition used in the present invention in a concentration of from 0.5 to 20 wt %, and preferably from 1 to 15 wt %.

Conceivable inorganic UV filters are those selected from the group comprising titanium dioxides, eg, coated titanium dioxide (eg, Eusolex® T 2000 or Eusolex® T-Aqua), zinc oxides (eg, Sachtotec®), iron oxides or, alternatively, cerium oxides. These inorganic UV filters are usually employed in the topical composition used in the present invention in a concentration of from 0.5 to 20 wt %, and preferably from 2 to 10 wt %.

Preferred UV filters are zinc oxide, titanium dioxide, 3-(4′-methylbenzylidene)-dl-camphor, 1-(4-tert-butylphenyl)-3-(4-methoxyphenyl)propane-1,3-dione, 4-isopro-pyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazol-5-sulfonic acid, and the potassium, sodium, and triethanolamine salts thereof.

Particularly preferred UV filters are zinc oxide and titanium dioxide.

If titanium dioxide is used in the present invention, there are preferably used, in addition to titanium dioxide, one or more further UV filters, selected from the group comprising 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazol-5-sulfonic acid, and the potassium, sodium, and triethanolamine salts thereof.

It is particularly preferred to use, in addition to titanium dioxide, the UV filters 2-hydroxy-4-methoxybenzophenone and/or 2-ethylhexyl p-methoxycinnamate.

In the present invention, ectoin or ectoin derivatives can be used prophylactically, ie in the absence of stress, or when stress is apparent. The use of ectoin or ectoin derivatives as proposed in the present invention produces a higher concentration of stress proteins under normal conditions, and under conditions of stress. Thus a reduction in the concentration of stress proteins in the skin can be effectively avoided. Furthermore, the synthesis of stress proteins is stimulated by the use of ectoin or ectoin derivatives as proposed in the present invention. In all, there is an improvement in the cell defence mechanism toward stress factors.

The following formulation examples illustrate the present invention. All compounds or ingredients that can be used in the cosmetic formulations are either known and commercially available or can be synthesized by known methods.

The INCI names of the starting materials used are as follows (the INCI names are, by way of definitaion, always stated in the English language):

| STARTING | |
|---|---|
| MATERIAL | INCI-NAME |
| Almond oil | Sweet Almond Oil (Prunus Dulcis) |
| Eutanol G | Octyldodecanol |
| Luvitol EHO | Cetearyl Octanoate |
| Oxynex K liquid | PEG-8, Tocopherol, Ascorbyl Palmitate, Ascorbic |
| | Acid, Citric Acid |
| Panthenol | Panthenol |
| Karion F liquid | Sorbitol |
| Sepigel 305 | Polyacrylamide, C13-14 Isoparaffin, Laureth-7 |
| Paraffin, liquid | Mineral Oil (Paraffinum Liquidum) |
| Mirasil CM 5 | Cyclomethicone |
| Arlacel 165 | Glyceryl Stearate, PEG-100 Stearate |
| Germaben II | Propylene Glycol, Diazolidinyl Urea, |
| | Methylparaben, Propylparaben |
| Perfume Bianca | Parfum |
| Abil WE 09 | Polyglyceryl-4 Isostearate, Cetyl Dimethicone |
| | Copolyol, Hexyl Laurate |
| Jojoba oil | Jojoba Oil (Buxus Chinensis) |
| Cetiol V | Decyl Oleate |
| Prisorine IPIS 2021 | Isopropyl Isostearate |
| Castor oil | Castor Oil (Ricinus Communis) |
| Lunacera M | Cera Microcristallina |
| Miglyol 812 neutral oil | Caprylic/Capric Triglyceride |
| Eusolex T-2000 | Titanium Dioxide, Alumina, Simethicone |

### EXAMPLE 1

The following ingredients are formulated as follows to produce a skin-care gel (O/W) containing ectoin:

| | | Wt % |
|---|---|---|
| A) Almond oil | (2) | 8.0 |
| Eutanol G | (3) | 2.0 |
| Luvitol EHO | (4) | 6.0 |
| Oxynex K liquid (Art. No. 108324) | (1) | 0.05 |
| B) Panthenol (Art. No. 501375) | (1) | 0.5 |
| Karion F liquid (Art. No. 102993) | (1) | 4.0 |
| Preservative | | q.s. |
| Water, demineralized | | ad 100 |
| C) Sepigel 305 | (5) | 3.0 |
| D) Ectoin | (1) | 1.0 |

The following compounds may be used as preservative:

0.05% propyl 4-hydroxybenzoate (Art. No. 107427) or

0.15% methyl 4-hydroxybenzoate (Art. No. 106757).

Preparation:

The combined phase B is slowly added to phase C with stirring. Predissolved phase A is then added. The mixture is stirred until the phases are mixed to a homogenous mass. Phase D is then added, and stirring is continued until a homogeneous composition is obtained.

Sources of Supply:

(1) Merck KGaA, Darmstadt

(2) Gustav Heess, Stuttgart

(3) Henkel KGaA, Dusseldorf

(4) BASF AG, Ludwigshafen

(5) Seppic, Frankreich

### EXAMPLE 2

The following ingredients are formulated as follows to produce a skin-care cream (O/W) containing ectoin:

| | | Wt % |
|---|---|---|
| A) Paraffin, liquid (Art. No. 107174) | (1) | 8.0 |
| Isopropyl myristate (Art. No. 822102) | (1) | 4.0 |
| Mirasil CM 5 | (2) | 3.0 |
| Stearic acid | (1) | 3.0 |
| Arlacel 165 | (3) | 5.0 |
| B) Glycerin. 87% (Art. No. 104091) | (1) | 3.0 |
| Germaben II | (4) | 0.5 |
| Water, demineralized | | ad 100 |
| C) Perfume Bianca | (5) | 0.3 |
| D) Ectoin | (1) | 1.0 |

Preparation:

Phases A and B are first of all separately heated to 75° C. Phase A is then slowly added to phase B with stirring, and stirring is continued until a homogeneous mixture is formed. Following homogenization of the emulsion, the latter is cooled to 30° C. with stirring and phases C and D are added, and stirring is continued until a homogeneous composition is obtained.

Sources of Supply:

(1) Merck KGaA, Darmstadt

(2) Rhodia

(3) ICI

(4) ISP

(5) Dragoco

### EXAMPLE 3

The following ingredients are formulated as follows to produce a sunscreen lotion (O/W) containing ectoin:

| | | Wt % |
|---|---|---|
| A) Abil WE 09 | (2) | 5.0 |
| Jojoba oil | (3) | 6.0 |
| Cetiol V | (4) | 6.0 |
| Prisorine 2021 | (5) | 4.5 |
| Castor oil | (6) | 1.0 |
| Lunacera M | (7) | 1.8 |
| Miglyol 812 neutral oil | (8) | 4.5 |
| B) Eusolex T-2000 (Art. No. 105373) | (1) | 3.0 |
| Glycerin, 87% (Art. No. 104091) | (1) | 2.0 |
| Sodium chloride (Art. No. 106400) | (1) | 0.4 |
| Preservative | | q.s. |
| Water, demineralized | | ad 100 |
| C) Perfume | (5) | 0.3 |
| D) Ectoin | (1) | 1.0 |

The following compounds may be used as preservatives:

0.05% propyl 4-hydroxybenzoate (Art.No. 107427) or

0.15% methyl 4-hydroxybenzoate (Art.No. 106757)

Preparation:

First Eusolex T 2000 is stirred into phase B and the mixture is heated to 80° C. Phase A is then heated to 75° C. and phase B slowly added with stirring. Stirring is continued until a homogeneous composition is obtained, and the mixture is then cooled to 30° C. with stirring. Phases C and D are then added, and stirring is continued until a homogeneous composition is obtained.

Sources of Supply:

(1) Merck KGaA, Darmstadt

(2) Th. Goldschmidt AG, Essen

(3) H. Lamotte, Bremen

(4) Henkel KGaA, Düsseldorf

(5) Unichema, Emmerich

(6) Gustav Heess, Stuttgart

(7) H. B. Fuller, Lüneburg

(8) Hüls Troisdorf AG, Witten

### EXAMPLE 4

The following ingredients are formulated as follows to produce a skin-care cream (O/W) containing ectoin:

| | | Wt. % |
|---|---|---|
| A) Paraffin, liquid (Art. No. 107174) | (1) | 8.0 |
| Isopropyl myristate (Art. No. 822102) | (1) | 4.0 |
| Mirasil CM 5 | (2) | 3.0 |
| Stearic acid | (1) | 3.0 |
| Arlacel 165 V | (3) | 5.0 |
| B) Glycerin, 87% (Art. No. 104091) | (1) | 3.0 |
| Germaben II | (4) | 0.5 |
| Water, demineralized | | ad 100 |
| C) Ectoin | (1) | 2.5 |

Preparation:

Phases A and B are first of all separately heated to 75° C. Phase A is then slowly added to phase B with stirring, and the mixture is stirred until a homogeneous composition is formed. Following homogenization of the emulsion, the mixture is cooled to 30° C. with stirring. Phase D is added, and stirring is continued until a homogeneous composition is obtained.

Sources of Supply:

(1) Merck KGaA, Darmstadt

(2) Rhodia

(3) ICI

(4) ISP

### EXAMPLE 5

Hair Tonic Containing Ectoin

| Starting material | | INCI-Name | Wt % |
|---|---|---|---|
| MERCARE ® Biotin | (1) | Biotin | 0.05 |
| Art. No. 130220 | | | |
| MERCARE ® Ectoin | | (Ectoin) | 1.00 |
| Art. No. 130200 | | | |
| Octopirox | (2) | Piroctone Olamine | 0.10 |
| D(+)panthenyl alcohol | (3) | Panthenol | 0.30 |
| (Art. No. 501375) | | | |
| Salicylic acid | (1) | Salicylic Acid | 0.10 |
| (Art. No. 100631) | | | |
| N-Cetyl-N.N.N-trimethyl- | (1) | Cetrimonium Bromide | 0.10 |
| ammonium bromide | | | |
| (Art. No. 102343) | | | |
| Dragoplant Hamamelis | (4) | Aqua. Alcohol Dentat. | 1.00 |
| | | Hamamelis Virginiana | |
| Isopropyl alcohol | (1) | Isopropyl Alcohol | 45.00 |
| (Art. No. 100995) | | | |
| Demineralized water | | Aqua | ad 100 |

Preparation:

Biotin was dissolved in the water and isopropyl alcohol. Ectoin was then dissolved, and the remaining starting materials were added with stirring.

Sources of Supply:

(1) Merck KGaA

(2) Hoechst

(2) BASF

(3) Dragoco

### EXAMPLE 6

2 in 1 Shampoo

| Starting material | | INCI-Name | Wt % |
|---|---|---|---|
| Jaguar C-162 | (2) | Hydroxypropyl | 0.20 |
| | | Guar | |
| | | Hydroxypropyltrimonium | |
| | | Chloride | |
| Miranol Ultra C32 | (2) | Sodium Cocoamphoacetate | 10.00 |
| Texapon NSO | (3) | Sodium Laureth Sulfate | 32.00 |
| Nicotinamide (vitamin B3) | (1) | Niacinamide | 0.10 |
| (Art. No. 130179) | | | |
| (D+)-biotin (vitamin H) | (1) | Biotin | 0.05 |
| (Art. No. 130220) | | | |
| MERCARE ® Ectoin | (1) | (Ectoin) | 1.00 |
| (Art. No. 130200 | | | |
| D-panthenol | (4) | Panthenol | 0.50 |
| Sodium chloride | (1) | Sodium Chloride | 1.0 |
| (Art. No. 106400) | | | |
| Perfume | | Parfum | |
| Preservative | | | q.s. |
| Citric acid | (1) | Citric acid | q.s. |
| (Art. No. 130137) | | | |
| Demineralized water | | Aqua | ad 100 |

Preparation:

Jaguar C 162 was dispersed in water and hydrated with citric acid. The remaining starting materials were added in the order given with stirring. The viscosity was then adjusted with NaCl and the pH with citric acid.

Sources of Supply:

(1) Merck KGaA

(2) Rhodia

(3) Cognis GmbH

(4) BASF AG

### EXAMPLE 7

Hair Styling Gel

| | | | |
|---|---|---|---|
| Starting material | Art. No. | INCI-Name | Wt % |

A

| | | | | |
|---|---|---|---|---|
| Pearlescent pigment | | (1) | | 1.00 |
| Carbopol ETD 2001 | | (2) | Carbomer | 0.50 |
| Isopropyl alcohol z.A. | 1.09634 | (1) | Isopropyl Alcohol | 20.00 |
| Water, demineralized | | | Aqua (Water) | 30.00 |

B

| | | | | |
|---|---|---|---|---|
| Luviskol K 30 Pulver | | (3) | PVP | 1.60 |
| Germaben II | | (4) | Propylene Glycol, | 0.20 |
| | | | Diazolidinyl | |
| | | | Urea, | |
| | | | Methylparaben, | |
| | | | Propylparaben | |
| Triethanolamine, high- | 108377 | (1) | Triethanolamine | 1.20 |
| grade | | | | |
| MERCARE ® | 130200 | (1) | (Ectoin) | 1.00 |
| ECTOIN | | | | |
| Water, demineralized | | | Aqua (Water) | 45.60 |

Preparation:

The pearlescent pigment was dispersed in the water/propanol mixture of phase A and the Carbopol was disseminated with stirring. Following complete dissolution, the predissolved phase B was slowly stirred in.

Note:

Recommended pearlescent pigments are interference pigments, silver pigments, gold pigments, and iron oxide pigments.

Sources of Supply:

(1) Merck KGaA

(2) BF Goodrich GmbH

(3) BASF AG

(4) ISP Global Technologies

### EXAMPLE 8

Syndet Soap

| Starting material | | INCI-Name | Wt % |
|---|---|---|---|
| Zetasap 813 A | (2) | Disodium Lauryl Sulfosuccinate, | 90.0 |
| | | Sodium Cocoyl Isothionate, | |
| | | Cetearyl Alcohol, Corn Starch, | |
| | | Glyceryl Stearate, Paraffin, | |
| | | Titanium Dioxide | |
| Ectoin | (1) | (Ectoin) | 1.00 |
| (Art. No. 130200) | | | |
| Perfume | | Parfum | 1.00 |
| Demineralized water | | Aqua (Water) | 8.00 |

Sources of Supply:

(1) Merck KGaA

(2) Zschimmer & Schwarz

### EXAMPLE 9

Shower gel

| | | | |
|---|---|---|---|
| Starting | | | |
| material | Art. No. | INCI-Name | Wt % |

A

| | | | | |
|---|---|---|---|---|
| Timiron | 1.17477 | (1) | CI 77891 (Titanium | 0.10 |
| Splendid | | | Dioxide), Mica, Silica | |
| Green | | | | |
| Keltrol T | | (2) | Xanthan Gum | 0.75 |
| Water, | | | Aqua (Water) | 62.10 |
| de- | | | | |
| mineralized | | | | |

B

| | | | | |
|---|---|---|---|---|
| Plantacare | | (3) | Decyl Glucoside | 20.00 |
| 2000 | | | | |
| Texapon | | (3) | Magnesium Oleth Sulfate, | 0.65 |
| ASV | | | Sodium Oleth Sulfate, | |
| | | | Magnesium Laureth-8 | |
| | | | Sulfate, | |
| | | | Sodium Laureth-8 Sulfate, | |
| | | | Magnesium Laureth | |
| | | | Sulfate, | |
| | | | Sodium Laureth Sulfate | |
| Bronidox L | | (3) | Propylene Glycol 5-bromo- | 0.20 |
| | | | 5-nitro-1,3-dioxane | |
| Perfume oil | | (4) | Parfum | 0.05 |
| Everest | | | | |
| 79658 SB | | | | |
| MERCARE ® | 130200 | (1) | (Ectoin) | 1.00 |
| Ectoin | | | | |

C

| | | | | |
|---|---|---|---|---|
| Citric acid | 130137 | (1) | Citric Acid | 0.15 |
| monohydrate | | | | |
| Water, | | | Aqua (Water) | 10.00 |
| demineralized | | | | |

Preparation:

To create phase A, the pigment was stirred into the water. Keltrol T was slowly disseminated with stirring and was stirred until dissolved. Phases B and C were added successively, and slow stirring was continued until all ingedients were homogeneously distributed.

Sources of Supply:

(1) Merck KGaA

(2) Kelco

(3) Cognis GmbH

(4) Haanmann & Reimer GmbH

### EXAMPLE 10

Baby powder

| | | | |
|---|---|---|---|
| Starting material | Art. No. | INCI-Name | Wt % |

A

| | | | | |
|---|---|---|---|---|
| IR 3535 TM | 111887 | (1) | Ethylbutylacetylaminopropionate | 4.00 |

B

| | | | | |
|---|---|---|---|---|
| Magnesium | 105827 | (1) | Magnesium Carbonate | 10.00 |
| carbonate | | | Hydroxdie | |
| hydroxide | | | | |
| Dry Flo PC | | (2) | Aluminum Starch | 86.00 |
| | | | Octenyl Succinate | |
| MERCARE ® | 130200 | (1) | (Ectoin) | 1.00 |
| Ectoin | | | | |

Preparation:

Phase B was placed in a vessel and mixed with a propeller stirrer. Phase A was added dropwise with stirring.

Sources of Supply:

(1) Merck KGaA

(2) National Starch & Chemical

### EXAMPLE 11

O/W After Sun Lotion

| | | | |
|---|---|---|---|
| Starting material | Art. No. | INCI-Name | Wt % |

A

| | | | | |
|---|---|---|---|---|
| MERCARE ® Bisabolol | 130170 | (1) | Bisabolol | 0.30 |
| Montanov 68 | | (2) | Cetearyl Alcohol, | 4.00 |
| | | | Cetearyl Glucoside | |
| Miglyol 812, neutral oil | | (3) | Caprylic/Capric | 12.00 |
| | | | Triglyceride | |
| Mirasil CM5 | | (4) | Cyclomethicone | 2.00 |
| Mirasil DM 350 | | (4) | Dimethicone | 1.00 |

B

| | | | | |
|---|---|---|---|---|
| Water, demineralized | | | Aqua (Water) | 77.20 |
| Glycerin (87% high-grade) | 104091 | (1) | Glycerin | 3.00 |
| Preservative | | | | q.s. |
| MERCARE ® Ectoin | 130200 | (1) | (Ectoin) | 1.00 |

C

| | | | |
|---|---|---|---|
| Rhodicare-S | (4) | Xanthan Gum | 0.50 |

Preparation:

Phases A and B were separately heated to 75° C., and phase C was added slowly to phase B at 75° C. with stirring, and the mixture was stirred until homogeneous. Phase A was then added to the mixture B/C and the whole homogenized. With stirring, the resulting mixture was cooled to room temperature.

Sources of Supply:

(1) Merck KGaA

(2) Seppic

(3) Hüls AG

(4) Rhodia GmbH

### EXAMPLE 12

Sunscreen Lotion (W/O)

| | | | |
|---|---|---|---|
| Starting | | | |
| material | Art. No. | INCI-Name | Wt % |

A

| | | | | |
|---|---|---|---|---|
| Eusolex | 105385 | (1) | 4-Methylbenzylidene | 4.00 |
| 8300 | | | Camphor | |
| Eusolex | 105382 | (1) | Octyl Methoxycinnamate, | 7.00 |
| 2292 | | | BHT | |
| Abil | | (2) | Polyglyceryl-4-isostearate, | 5.00 |
| WE 09 | | | Cetyl Dimethicone | |
| | | | Copolyol, | |
| | | | Hexyl Laurate | |
| Jojoba oil | | (3) | Buxus Chinensis (Jojoba | 3.00 |
| | | | Oil) | |
| Cetiol V | | (4) | Decyloleate | 3.00 |
| Prisorine | | (5) | Isopropyl Isostearate | 2.00 |
| 2021 | | | | |
| Paracera | | (6) | Microwax | 1.00 |
| M | | | | |
| Miglyol | | (7) | Caprylic/Capric | 3.00 |
| 812, | | | Triglyceride | |
| neutral oil | | | | |
| Propyl | 1.07427 | (1) | Propylparaben | 0.05 |
| 4- | | | | |
| hydroxy- | | | | |
| benzoate | | | | |

B

| | | | | |
|---|---|---|---|---|
| Eusolex | 105401 | (1) | Aqua (Water), | 16.00 |
| T-Aqua | | | Titanium Dioxide, | |
| | | | Alumina, | |
| | | | Sodium Metaphosphate, | |
| | | | Phenoxyethanol, Sodium | |
| | | | Methylparaben | |
| Glycerin | 104091 | (1) | Glycerin | 2.00 |
| (87% | | | | |
| high- | | | | |
| grade) | | | | |
| Sodium | 106400 | (1) | Sodium Chloride | 0.40 |
| chloride | | | | |
| MERCARE ® | 130200 | (1) | (Ectoin) | 1.00 |
| Ectoin | | | | |
| Water, | | | Aqua (Water) | 53.40 |
| demineralized | | | | |
| Methyl | 106757 | (1) | Methylparaben | 0.15 |
| 4- | | | | |
| hydroxy- | | | | |
| benzoate | | | | |

Preparation:

Phase B was heated to 80° C. and Phase A was heated to 75° C. Phase B was slowly stirred into Phase A. The mixture was homogenized and cooled with stirring.

Sources of Supply:

(1) Merck KGaA

(2) Th. Goldschmidt AG

(3) Henry Lamotte GmbH

(4) Cognis GmbH

(5) Unichema Chemie GmbH

(6) Paramelt

(7) Hüls AG

### EXAMPLE 13

Tooth Gel

| | | | |
|---|---|---|---|
| Starting material | Art. No. | INCI-Name | Wt % |

A

| | | | | |
|---|---|---|---|---|
| Sodium fluoride | 106441 | (1) | Sodium Fluoride | 0.06 |
| Karion F liquid | 152698 | (1) | Sorbitol | 48.39 |
| Sodium benzoate | 106290 | (1) | Sodium Benzoate | 0.16 |
| Sodium saccharinate | | | | 0.16 |
| MERCARE ® Ectoin | 130200 | (1) | (Ectoin) | 1.00 |
| Water, demineralized | | | Aqua (Water) | 29.12 |

B

| | | | | |
|---|---|---|---|---|
| MERCARE ® Olaflur | 111680 | (1) | Olaflur, Propylene | 1.17 |
| | | | Glycol | |
| Bromochlorophene | 1.03281 | (1) | Bromochlorophene | 0.08 |
| Aroma 35049 | | (2) | | 0.78 |

C

| | | | | |
|---|---|---|---|---|
| Polyethylene | 807485 | (1) | PEG-8 | 2.34 |
| glycol 400 | | | | |
| Tego Betain ZF | | (3) | Cocamidopropyl | 3.89 |
| | | | Betaine | |
| Sicomet Patent Blue | | (4) | | 0.62 |
| (E131), 0.1% in water | | | | |

D

| | | | |
|---|---|---|---|
| Sident 12 | (5) | Silica | 7.40 |
| Sipemat 22 S | (5) | Hydrated Silica | 5.84 |

Preparation:

Phases A and B were premixed separately. Phase C was heated to 50° C. Phases A and B were stirred into phase C and the mixture was stirred in vacuo. Following the slow addition of phase D, the mixture was homogenized in vacuo. Stirring was continued in vacuo until the gel had a clear appearance.

Sources of Supply:

(1) Merck KGaA

(2) Crissa Drebing GmbH

(3) Th. Goldschmidt AG

(4) BASF AG

(5) Degussa AG

### EXAMPLE 14

Mouth Wash Concentrate

| Starting material | | Wt % |
|---|---|---|
| MERCARE ® Ectoin | (1) | 1.00 |
| N-Cetylpyridinium chloride (Art. No. 102340) | (1) | 0.50 |
| Ethanol (96%) (Art. No. 100971) | (1) | 70.00 |
| Peppermint aroma 77526-34 | (2) | 0.15 |
| Water, demineralized | | ad 100.00 |

Preparation:

All ingredients were stirred together until a clear solution was obtained.

Sources of Supply:

(1) Merck KGaA

(2) Givaudan-Roure, Dortmund

### EXAMPLE 15

Lip Balsam

| Starting material | | INCI-Name | Wt % |
|---|---|---|---|
| Ectoin | (1) | (Ectoin) | 1.00 |
| (Art. No. 130200) | | | |
| Tagat S2 | (2) | PEG-20 Glyceryl Stearate | 10.00 |
| Lanette O | (3) | Cetearyl Alcohol | 20.00 |
| Glycerin (87%) | (1) | Glycerin | 20.00 |
| (Art. No. 104091) | | | |
| Vaseline | (4) | Petrolatum | 35.00 |

Preparation

All ingredients were heated to 75° C., and the mixture was then cooled to room temperature with stirring.

Sources of Supply:

(1) Merck KGaA

(2) Goldschmidt GmbH

(3) Cognis GmbH

(4) Schumann Sasol

### EXAMPLE 16

Lip Gloss

| | | | |
|---|---|---|---|
| Starting material | Art. No. | INCI-Name | Wt % |

A

| | | |
|---|---|---|
| Pearlescent | (1) | 10.00 |
| pigments | | |

B

| | | | | |
|---|---|---|---|---|
| Indopol H 100 | | (2) | Polybutene | 59.95 |
| Bentone Gel MIO V | | (3) | Quaternium-18 | 20.00 |
| | | | Hectorite, | |
| | | | Propylene Carbonate | |
| | | | Paraffinum Liquidum | |
| | | | (Mineral Oil) | |
| Eutanol G | | (4) | Octyldodecanol | 6.00 |
| MERCARE^{®} | 130180 | (1) | Tocopheryl Acetate | 1.00 |
| Tocopherl acetate | | | 1.00 | |
| Dow Corning 1403 | | (5) | Dimethiconol, | 3.00 |
| Fluid | | | Dimethicone, | |
| Propyl 4- | 1.07427 | (1) | Propylbarabene | 0.05 |
| hydroxybenzoate | | | | |

C

| | | | |
|---|---|---|---|
| MERCARE ® | (1) | (Ectoin) | 1.00 |
| Ectoin | | | |

Preparation:

All of the ingredients of phase B were weighed in, heated (60-70° C.) and thoroughly stirred until a homogeneous composition resulted. Phases B and C were then added, and the mixture was again stirred well. The homogeneous mixture was bottled at 50-60° C.

Sources of Supply:

(1) Merck KGaA

(2) Amoco

(3) Rheox

(4) Cognis GmbH

(5) Dow Corning

### EXAMPLE 17

Lip Herpes Cream

| Starting material | | INCI-Name | Wt % |
|---|---|---|---|
| Ectoin | (1) | (Ectoin) | 1.00 |
| (Art. No. 130200) | | | |
| Aciclovir | | (9-[(2-Hydroxyethoxy)- | 5.00 |
| | | methyl]guanin) | |
| Tagat S2 | (2) | PEG-20 Glyceryl Stearate | 10.00 |
| Lanette O | (3) | Cetearyl Alcohol | 20.00 |
| Glycerin (87%) | | Glycerin | 20.00 |
| (Art. No. 104091) | | | |
| Vaseline | (4) | Petrolatum | 35.00 |
| Demin. water | | Aqua (Water) | ad 100 |

Preparation:

All ingredients were heated to 75° C., and the mixture was then cooled to room temperature with stirring.

Sources of supply:

(1) Merck KGaA

(2) Goldschmidt GmbH

(3) Cognis GmbH

(4) Schumann Sasol

Die topical compositions prepared in Examples 1 to 17 are for application to the skin to provide protection of the stress proteins.

### EXAMPLE 18

Hydrogel Containing Ectoin

| | | | |
|---|---|---|---|
| Starting material | Art. No. | INCI-Name | Wt % |

A

| | | | | |
|---|---|---|---|---|
| TIMIRON ® | 117474 | (1) | Cl 77891 (Titanium Dioxide), | 0.10 |
| Splendid Gold | | | Mica, Silica | |
| Carbopol Ultrez | | (2) | Carbomer | 0.40 |
| 10 | | | | |
| Water, | | | Aqua (Water) | 67.70 |
| demineralized | | | | |

B

| | | | | |
|---|---|---|---|---|
| RonaCare™ | 130200 | (1) | (Ectoin) | 1.00 |
| Ectoin | | | | |
| Tris- | 130132 | (1) | Tromethamine | 0.60 |
| (hydroxymethyl)- | | | | |
| aminomethane | | | | |
| Germaben II | | (3) | Propylene Glycol, Diazolidinyl, | 0.20 |
| | | | Urea, Methylparaben, | |
| | | | Propylparaben | |
| Water, | | | Aqua (Water) | 10.00 |
| demineralized | | | | |

C

| | | | |
|---|---|---|---|
| Lubrajel DV | (4) | Propylene Glycol, Polyglyceryl | 18.00 |
| | | Methacrylate | |
| D | (4) | PVM/MA Copolymer, | |
| Lubrajel Oil | | Proplyene | |
| | | Glycol, Glyceryl | 2.00 |
| | | Polymethacrylate | |

Preparation:

The pearlescent pigment was dispersed in the water of phase A and the Carbopol was added with stirring. Following complete dissolution, the predissolved phase B was stirred in. Finally, phases C and D were added.

Comments:

Opaque, lustrous gold gel

| | |
|---|---|
| pH (25° C.): | 6.5 |
| Viscosity (25° C.): | 60 000 mPa · s (Brookfield RVT, |
| | spindle C, 5 rpm, Helipath) |

Sources of Supply:

(1) Merck KGaA

(2) BF Goodrich GmbH

(3) ISP Global Technologies

(4) Guardian

### EXAMPLE 19

After-Shave Soft Cream

| | | | |
|---|---|---|---|
| Starting material | Art. No. | INCI-Name | Wt % |

A

| | | |
|---|---|---|
| Eumulgin B1 | (1) Ceteareth-12 | 0.50 |
| Eumulgin B2 | (2) Ceteareth-20 | 0.50 |
| Cutina MD-V | (1) Glyceryl Stearate | 3.00 |
| Cetiol LC | (1) Coco-caprylate | 5.00 |
| | Caprate | |
| Carbopol Ultrez 10 | (2) Carbomer | 0.30 |

B

| | | | |
|---|---|---|---|
| Water, demineralized | | Aqua (Water) | 66.10 |
| Glycerin (87% high-grade) | 104091 | (3) Glycerin | 3.00 |
| Ethanol (96% high-grade) | 100971 | (3) Alcohol | 20.00 |
| Menthol, cryst. | 105995 | (3) Menthol | 0.30 |
| Tris(hydroxymethyl)amino- | 130132 | (3) Tromethamine | 0.30 |
| methane | | | |
| RonaCare ™ Ectoin | 130200 | (Ectoin) | 1.00 |

Preparation:

Phases A and B were separately heated to 80° C. Phase A was added to phase B with stirring and the mixture was homogenized and then cooled to room temperature with stirring.

Comments:

| | |
|---|---|
| pH (25° C.): | 7.25 |
| Viscosity (25° C.): | 34 000 mPa · s (Brookfield RVT, spindle C, 5 rpm, |
| | Helipath) |

Sources of Supply:

(1) Cognis GmbH

(2) BF Goodrich GmbH

(3) Merck KGaA

### EXAMPLE 20

Evening Cream Containing RonaCare ™ Ectoin

| | | | |
|---|---|---|---|
| | | | Wt. |
| Starting material | Art. No. | INCI-Name | % |

A

| | | | |
|---|---|---|---|
| TIMIRON ® Splendid | 117474 | (1) Cl 77891 (Titanium | 2.00 |
| Gold | | Dioxide), | |
| | | Mica, Silica | |
| Carbopol ETD 2001 | | (2) Carbomer | 0.50 |
| Citric acid | 102895 | (1) Citric acid | q.s. |
| Water, demineralized | | Aqua (Water) | 40.00 |

B

| | | | |
|---|---|---|---|
| Propylene glycol | 107478 | (1) Propylene glycol | 3.00 |
| RonaCare ™ Ectoin | 130200 | (1) (Ectoin) | 1.00 |
| Water, demineralized | | Aqua (Water) | 28.45 |
| Preservative | | | |

C

| | | | |
|---|---|---|---|
| Hostaphat KL 340 N | | (3) Dilaureth-4 Posphate | 3.00 |
| Cetyl alcohol | 100989 | (1) Cetyl Alcohol | 2.00 |
| Paraffin liquid | 107162 | (1) Paraffinum Liquidum | 10.00 |
| | | (Mineral Oil) | |
| Cetiol V | | (4) Decyl Oleate | 6.00 |

D

| | | | |
|---|---|---|---|
| Triethanolamine high-grade | 108377 | (1) Triethanolamine | 0.35 |
| Water, demineralized | | Aqua (Water) | 3.50 |

E

| | | | |
|---|---|---|---|
| Perfume Vogue | 2309334 | (5) Parfum | 0.20 |

Preparation:

The pearlescent pigment was dispersed in the water of phase A. The mixture was possibly acidified with some drops of citric acid in order to reduce the viscosity. Carbopol was disseminated with stirring. Following complete dissolution, the predissolved phase B was slowly stirred in. Phase A/B and phase C were heated to 80° C., phase C was stirred into phase A/B, and the mixture was homogenized, neutralized with phase D, again homogenized, and cooled with stirring. Comments:

| | |
|---|---|
| pH (24° C.): | 5.8 |
| Viscosity: | 33000 mPa · s (Brookfield RVT, spindle C, |
| | 5 rpm, Helipath), 24 ° C. |

### EXAMPLE 21

Pre-Solarium Soft Cream Containing RonaCare ™ Ectoin

| | | | |
|---|---|---|---|
| | | | Wt. |
| Starting material | Art. No. | INCI-Name | % |

A

| | | | |
|---|---|---|---|
| Paraffin viscous | 107160 | (1) Paraffinum Liquidum (Mineral | 10.00 |
| | | Oil) | |
| Cetiol S | | (2) Dioctylcyclohexane | 2.50 |
| Isopropyl palmitate | | (2) Isopropyl Palmitate | 6.50 |
| Miglyol 812 N | | (3) Caprylic/Capric Triglyceride | 1.00 |
| Sunflower seed oil | | (4) Helianthus Annuus (Sunflower | 5.00 |
| | | Seed Oil) | |
| OXYNEX ® | 108324 | (1) PEG-8, Tocopherol, Ascorbyl | 0.10 |
| K liquid | | Palmitate, Ascorbic Acid, | |
| | | Citric Acid | |

B

| | | |
|---|---|---|
| Carbopol ETD 2001 | (5) Carbomer | 0.30 |

C

| | | | |
|---|---|---|---|
| Water, | | Aqua (Water) | 68.40 |
| demineralized | | | |
| RonaCare ™ Ectoin | 130200 | (1) (Ectoin) | 1.00 |
| Sisterna L70-C | | (6) Aqua (Water), Sucrose Laurate, | 5.00 |
| | | Alcohol | |
| Sodium hydroxide, | 105588 | (1) Sodium Hydroxide | 0.00 |
| 10% strength | | | |
| Preservative | | | 0.00 |

D

| | | |
|---|---|---|
| Perfume oil Nikita | (7) Parfum | 0.20 |

Preparation:

Phase B was dispersed in phase A. Predissolved phase C was added to phase A/B with stirring, and the mixture was neutralized and homogenized, and phase D was added with stirring.

Comments:

| | |
|---|---|
| pH (25° C.): | 5.5-6.5 |
| Viscosity: | 113000 mPa · s (Brookfield RVT, spindle C, 10 rpm, |
| | Helipath), 25° C. |

Sources of Supply:

(1) Merck KGaA

(2) Cognis GmbH

(3) Condea Chemie GmbH

(4) Gustav Heess GmbH

(5) BF Goodrich GmbH

(6) Sisterna C.V./ Dai-Ichi

(7) Dragoco

### EXAMPLE 22

Rich Night Cream Containing RonaCare ™ Ectoin

| | | | |
|---|---|---|---|
| Starting material | Art. No. | Inci | Wt % |

A

| | | |
|---|---|---|
| Isolan Gl 34 | (1) Polyglyceryl-4-isostearate | 1.00 |
| Abil EM 90 | (1) Cetyl Dimethicone Copolyol | 2.00 |
| Paracera W 80 | (2) Ceresin (Microcrystalline | 1.50 |
| | Wax) | |
| Cutina HR | (3) Hydrogenated Castor Oil | 0.50 |
| Cetiol V | (3) Decyl Oleate | 10.00 |
| Dragoxat EH | (4) Octyl Octanoate | 5.00 |
| Miglyol 812 N | (5) Caprylic/Capric Triglyceride | 10.00 |

B

| | | | |
|---|---|---|---|
| Glycerin (87% high- | 104091 | (6) Glycerin | 2.00 |
| grade) | | | |
| Magnesium sulfate | 105882 | (6) Magnesium Sulfate | 1.00 |
| heptahydrate | | | |
| RonaCare ™ Ectoin | 130200 | (6) (Ectoin) | 1.00 |
| Water, | | Aqua (Water) | 66.00 |
| demineralized | | | |
| Preservative | | | |

C

| | |
|---|---|
| Perfume oil | q.s. |

Preparation:

Phase A and phase B were heated separately to 80° C. Phase B was added with stirring to phase A. The mixture was homogenized, and phase C was added at ca 35° C. The mixture was then cooled to room temperature.

Comments:

| | |
|---|---|
| Viscosity: | 6500 mPa · s (Brookfield RVT, spindle C, 20 rpm, Helipath), |
| | 25° C. |

Sources of Supply:

(1) Th. Goldschmidt AG

(2) Paramelt

(3) Cognis GmbH

(4) Dragoco Gerberding & Co. AG

(5) Condea Chemie GmbH

(6) Merck KGaA

### EXAMPLE 23

Skin-care Cream containing RonaCare ™ Ectoin

| | | | |
|---|---|---|---|
| | | | Wt. |
| Starting material | Art. No. | INCI-Name | % |

A

| | | | |
|---|---|---|---|
| Paraffin viscous | 107160 | (1) Paraffinum Liquidum (Mineral | 8.00 |
| | | Oil) | |
| Tego Care 150 | | (2) Glyceryl Stearate, Steareth-25, | 10.00 |
| | | Ceteth-20, Stearyl Alcohol | |
| Lanette O | | (3) Cetearyl Alcohol | 1.50 |
| Isopropyl palmitate | | (3) Isopropyl Palmitate | 5.00 |
| Abil Wax 2434 | | (2) Stearoxy Dimethicone | 1.60 |
| Miglyol 812 N | | (4) Caprylic/capric Triglyceride | 2.00 |
| Dow Corning 200 | | (5) Dimethicone | 0.30 |
| Fluid (350 cs) | | | |

B

| | | | |
|---|---|---|---|
| Glycerin (87%) | 104091 | (1) Glycerin | 3.00 |
| RonaCare ™ Ectoin | 130200 | (6) (Ectoin) | 1.00 |
| Water, | | Aqua (Water) | 67.60 |
| demineralized | | | |
| Preservative | | | |

C

| | |
|---|---|
| Perfume oil | q.s. |

Preparation:

Phase A was heated to 75° C. Phase B was heated to 80° C. and then added to phase A with stirring. The mixture was homogenized, and phase C was added at ca 35° C. The mixture was cooled to room temperature with stirring.

Comments:

| | |
|---|---|
| pH (25° C.): | 5.1 |
| Viscosity: | 342000 mPa · s (Brookfield RVT, spindle C, 2.5 rpm, |
| | Helipath), at 24° C. |

Sources of Supply:

(1) Merck KGaA

(2) Th. Goldschmidt AG

(3) Cognis GmbH

(4) Condea Chemie GmbH

(5) Dow Corning

### EXAMPLE 24

Refreshing Cream Containing RonaCare ™ Ectoin (W/O)

| | | | |
|---|---|---|---|
| | | | Wt |
| Starting material | Art. No. | INCI-Name | % |

A

| | | | |
|---|---|---|---|
| Paraffin liquid | 107162 | (1) Paraffinum Liquidum (Mineral | 8.00 |
| | | Oil) | |
| Arlacel P135 | | (2) PEG-30 Dipolyhydroxystearate | 5.50 |
| Miglyol 812 N | | (3) Caprylic/Capric Triglyceride | 4.00 |
| Arlamol HD | | (2) Isohexadecane | 6.00 |

B

| | | | |
|---|---|---|---|
| Water, | | Aqua (Water) | 46.00 |
| demineralized | | | |
| Glycerin (87% high- | 104091 | (1) Glycerin | 4.00 |
| grade) | | | |
| RonaCare ™ Ectoin | 130200 | (1) (Ectoin) | 1.00 |
| Magnesium sulfate | 105882 | (1) Magnesium Sulfate | 0.50 |
| heptahydrate | | | |
| Preservative | | | |

C

| | | | |
|---|---|---|---|
| Ethanol 96% high- | 100971 | (1) Alcohol | 25.00 |
| grade | | | |

Preparation:

Phase A and phase B were separately heated to 75° C. Phase B was added to phase A with stirring. The mixture was homogenized, and phase C was added at ca 30° C. The mixture was cooled to room temperature with stirring.

Comments:

| | |
|---|---|
| Viscosity: | 41000 mPa · s (Brookfield RVT, spindle C, 5 rpm, Helipath), |
| | 24° C. |

Sources of Supply:

(1) Merck KGaA

(2) Uniqema

(3) Condea Chemie GmbH

### EXAMPLE 25

Skin-care Cream Containing RonaCare ™ Ectoin (W/O)

| | | | |
|---|---|---|---|
| | | | Wt |
| Starting material | Art. No. | INCI-Name | % |

A

| | | | |
|---|---|---|---|
| Paraffin viscous | 107160 | (1) Paraffinum Liquidum (Mineral | 10.00 |
| | | Oil) | |
| Hostacerin WO | | (2) Polyglyceryl-2-sesquiiso- | 6.00 |
| | | stearate, Cera Alba (Beeswax), | |
| | | Cera Microcristallina | |
| | | (Microcrystalline Wax), | |
| | | Paraffinum Liquidum (Mineral | |
| | | Oil), Magnesium Stearate, | |
| | | Aluminium Stearate | |
| Isopropyl palmitate | | (3) Isopropyl Palmitate | 8.00 |
| Paracera M | | (4) Microwax | 3.00 |
| Vaseline | | (5) Petrolatum | 3.00 |

B

| | | | |
|---|---|---|---|
| Water, | | (1) Aqua (Water) | 65.00 |
| demineralized | | | |
| Glycerin (87% high- | 104091 | (1) Glycerin | 4.00 |
| grade) | | | |
| RonaCare ™ Ectoin | 130200 | (Ectoin) | 1.00 |
| Preservative | | | |

Preparation:

Phase A and phase B were heated to 80° C. Phase B was added to phase A with stirring. The mixture was homogenized and cooled to room temperature with stirring.

Comments:

| | |
|---|---|
| Viscosity | 220000 mPa · s (Brookfield RVT, spindle D, 5 rpm, |
| | Helipath), at 24° C. |

Sources of Supply:

(1) Merck KGaA

(2) Clariant GmbH

(3) Cognis GmbH

(4) Paramelt

(5) Schümann Sabol

### EXAMPLE 26

W/O/W Night Cream Containing RonaCare ™ Ectoin

| | | | |
|---|---|---|---|
| Starting material | Art. No. | INCI-Name | Wt % |

A

| | | | |
|---|---|---|---|
| Brij 721 P | | (1) Steareth-21 | 2.00 |
| Brij 72 | | (1) Steareth-2 | 3.00 |
| Arlacel P135 | | (1) PEG-30 | 1.50 |
| | | Dipolyhydroxystearate | |
| Jojoba oil | | (2) Buxus Chinensis | 8.00 |
| | | (Jojoba Oil) | |
| RonaCare ™ Tocopheryl | 130180 | (3) Tocopheryl Acetate | 1.00 |
| acetate | | | |
| Lanette O | | (4) Cetearyl Alcohol | 1.00 |
| Stearic acid | 100671 | (3) Stearic Acid | 1.50 |
| Mirasil CM 5 | | (5) Cyclomethicone | 1.00 |

B

| | | | |
|---|---|---|---|
| Glycerin (87% high-grade) | 104091 | (3) Glycerin | 4.00 |
| Preservative | | | |
| RonaCare ™ Ectoin | 130200 | (Ectoin) | 1.00 |
| Water, demineralized | | Aqua (Water) | 76.00 |
| Sodium hydroxide, 10% | 105588 | (3) Sodium Hydroxide | |
| strength | | | |

Preparation:

Phase A and phase B were heated to 75° C. Phase A was slowly stirred into phase B. The mixture was homogenized, possibly neutralized with sodium hydroxide solution and cooled with stirring.

Comments:

| | |
|---|---|
| pH (22° C.): | 5.7 |
| Viscosity (24° C.): | 42000 mPa · s (Brookfield RVT, spindle C, 5 rpm, |
| | Helipath) |

Sources of Supply:

(1) Uniqema

(2) Gustav Heess GmbH

(3) Merck KGaA

(4) Cognis GmbH

(5) Rhodia GmbH

### EXAMPLE 27

Sprayable Sunscreen Lotion Containing RonaCare ™ Ectoin

| | | | |
|---|---|---|---|
| Starting material | Art. No. | INCI-Name | Wt % |

A

| | | | |
|---|---|---|---|
| EUSOLEX ® 2292 | 105382 | (1) Octyl Methoxycinnamate, | 7.50 |
| | | BHT | |
| EUSOLEX ® 4360 | 105376 | (1) Benzophenone-3 | 2.50 |
| EUSOLEX ® HMS | 111412 | (1) Homosalate | 7.00 |
| Hetester PHA | | (2) Propylene Glycol Isoceteth-3 | 5.00 |
| | | Acetate | |
| Volpo S-2 | | (3) Steareth-2 | 0.40 |
| Volpo S-10 | | (3) Steareth-10 | 0.80 |
| Pemulen TR-2 | | (4) Acrylates/C10-30 Alkyl | 0.18 |
| | | Acrylate Crosspolymer | |
| Performa V 825 | | (5) Synthetic wax | 0.80 |
| Dow Corning 200 | | (6) Dimethicone | 1.00 |
| (100 cs) | | | |
| OXYNEX ® K | 108324 | (1) PEG-8, Tocopherol, | 0.10 |
| | | Ascorbyl | |
| liquid | | palmitate, Ascorbic Acid, Citric | |
| | | Acid | |

B

| | | | |
|---|---|---|---|
| RonaCare ™ | 130200 | (1) (Ectoin) | 1.00 |
| Ectoin | | Aqua (water) | |
| Water, | | | 49.82 |
| demineralized | | | |
| Preservative | | | |

C

| | | | |
|---|---|---|---|
| Water, | | Aqua (Water) | 20.00 |
| demineralized | | | |
| EUSOLEX ® 232 | 105372 | (1) Phenylbenzimidazole | 1.00 |
| | | Sulfonic Acid | |
| Propylene glycol, | | (7) Propylene Glycol | 2.00 |
| Triethanolamine | 108377 | (1) Triethanolamine | 0.90 |
| high-grade | | | |

Preparation:

Phase A and phase B were heated to 80° C. Phase B was added to phase A with stirring, and the mixture was neutralized with phase C at room temperature and homogenized.

Comments:

| | |
|---|---|
| pH (21° C.): | 7.0 |
| Viscosity: | watery |

Sources of Supply:

(1) Merck KGaA

(2) Paroxite Ltd.

(3) Croda GmbH

(4) BF Goodrich GmbH

(5) New Phase Technologies

(6) Dow Corning

(7) Biesterfeld

### EXAMPLE 28

O/W Cream Containing RonaCare ™ Ectoin

| | | | |
|---|---|---|---|
| Starting material | Art. No. | INCI-Name | Wt % |

A

| | | |
|---|---|---|
| Tego Care 150 | (1) Glyceryl Stearate, | 8.00 |
| | Steareth-25, ceteth- | |
| | 20, Stearyl Alcohol | |
| Lanette 18 | (2) Stearyl Alcohol | 1.00 |
| Isopropyl palmitate | (2) Isopropyl Palmitate | 3.00 |
| Jojoba oil | (3) Buxus Chinensis | 7.00 |
| | (Jojoba Oil) | |

B

| | | | |
|---|---|---|---|
| RonaCare ™ | 130200 | (4) (Ectoin) | 1.00 |
| Glycerin (87% high-grade) | 104091 | (4) Glycerin | 3.00 |
| Preservative | | | |
| Water, demineralized | | Aqua (Water) | 77.00 |

Preparation:

Phases A and B were heated to 80° C. Phase B was added to phase A with stirring, and the mixture was homogenized and stirred until cold.

Comments:

| | |
|---|---|
| pH (23° C.): | 5.4 |
| Viscosity (24° C.): | 95000 mPa · s (Brookfield RVT, spindle C, 5 rpm, |
| | Helipath) |

Sources of Supply:

(1) GoldschmidtAG

(2) Cognis GmbH

(3) Gustav Heess GmbH

(4) Merck KGaA

### EXAMPLE 29

O/W Moisture Cream Containing RonaCare ™ Ectoin

| | | | |
|---|---|---|---|
| Starting material | Art. No. | INCI-Name | Wt % |

A

| | | | |
|---|---|---|---|
| RonaCare ™ Ectoin | 130200 | (1) Ectoin | 1.00 |
| Glycerin (87% high-grade) | 104091 | (1) Glycerin | 3.00 |
| Preservative | | | |
| Water, demineralized | | Aqua (Water) | 76.20 |

B

| | | | |
|---|---|---|---|
| Sisterna SP30-C | | (2) Sucrose Distearate | 2.70 |
| Sisterna SP70-C | | (2) Sucrose Stearate | 0.90 |
| Cetiol OE | | (3) Dicaprylyl Ether | 5.00 |
| Miglyol 812 | 106175 | (1) Caprylic/capric | 2.00 |
| | | Triglyceride | |
| Isopropyl palmitate | | (3) Isopropyl Palmitate | 2.00 |
| Cegesoft C 24 | | (3) Octyl Palmitate | 7.00 |
| Carbopol ETD 2001 | | (4) Carbomer | 0.20 |

C

| | | |
|---|---|---|
| Sodium hydroxide, 10% | 105588 | (1) Sodium Hydroxide |
| strength | | |

Preparation:

Phase A was heated to 75° C., and phase B was mixed well in the cold and then heated to 75° C. Phase B was then added to phase A with stirring, and the mixture was homogenized, neutralized and stirred until cold.

Comments:

| | |
|---|---|
| pH (22° C.): | 6.5 |
| Viscosity (21° C.): | 109000 mPa · s |
| | (Brookfield RVT, spindle C, 5 rpm, Helipath) |

Sources of Supply:

(1) Merck KGaA

(2) Sisterna C.V./ Dai-lchi

(3) Cognis GmbH

(4) BF Goodrich GmbH

### EXAMPLE 30

In vitro experiments have shown that human skin cells which have been treated with ectoin can protect themselves against stress factors significantly better than untreated skin cells. This was ascertained by spreading primary human keratinocytes on microscopic slides and cultivating them over a period of 3 days at 37° C. in KGM 2 medium (Cell Systems). The human keratinocytes were incubated to a confluence of approximately 60% and part of the culture was then treated with 1% ectoin based on the supernatant of the culture medium. The untreated portion of the culture served as control. Following treatment, the human keratinocytes were incubated for a further 16 hours at 37° C. The cultures were then subjected to heat shock by raising the temperature quickly from 37° C. to 44° C. The experiments were carried out by the method proposed by Holland et al in J. Invest Dermatol. (1993), 101: 196-199. Following this heat-shock treatment, incubation was carried out at 37° C. for a further 2 hours. The cells were then fixed. Fixing was carried out in 3 stages:

fixing solution A (1% CH₃COOH/40% ethanol in deionized water v/v, 4° C.), 5 min,

fixing solution B (96% ethanol in deionized water v/v, 4° C.), 5 min,

fixing solution A, 5 min.

The stress proteins produced were stained by immunofluorescence, which process involved the following steps:

blockage of non-specific bonding sites with a blocking agent (10% BSA in phosphate buffer): 45 min, 37° C.,

incubation with the primary antibody anti HSP 72/73 (1:200 dilution in phosphate buffer): 45 min, 37° C.,

incubation with the FITC-conjugated secondary antibody goat anti mouse (1:200 dilution in phosphate buffer): 45 min, 37° C.,

covering of the specimens with fluorescent covering medium and slide cover glasses.

The stess proteins were determined quantitatively as HSP 72/73 under a microscope (BX40, filter: WB, burner: U-RFL-T, Olympus).

The results of this analysis are shown in FIG. 2. As may be seen from the microscopic assessment of the cellular stress response of untreated cultures and ectoin-treated cultures displayed therein, preliminary treatment with ectoin gave rise to significantly faster induction of HSP 72/73 stress proteins than in the case of the untreated cultures. In particular, the ectoin-treated skin cells achieved an HSP 72/73 concentration maximum after a period of only 30 minutes, whilst in the case of the untreated skin cells the concentration of the HSP 72/73 stress proteins reached 100% only after a period of 60 minutes.

1. A method of using at least one compound selected from the group comprising compounds of formulas 1a and 1b 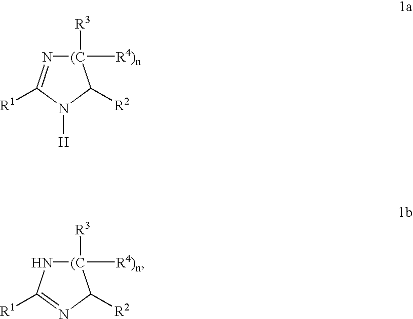
physiologically acceptable salts thereof, and stereoisomeric forms thereof, in which
R¹ denotes H oder alkyl,
R² denotes H, COOH, COO-alkyl or CO—NH—R⁵,
R³ and R⁴ each independently denote H or OH,
n is 1, 2 or 3,
R⁵ denotes H, alkyl, an amino acid group, a dipeptide group or a tfipeptide group, and
alkyl denotes an alkyl group containing from 1 to 4 carbons
for the protection of HSP 60, HSP 90 and HSP 72/73 in the skin.

2. A method as defined in claim 1, wherein said compound(s) are present in a topical composition.

3. A method as defined in claim 1 or claim 2, wherein at least one of the compounds defined in claim 1 is present in a topical composition in a concentration of from 0.0001 to 50 wt % based on the composition.

4. A method as defined in any one of claims 1 to 3, wherein (S)-1,4,5,6-tetrahydro-2-methyl-4-pyrimidinecarboxylic acid and/or (S,S)-1,4,5,6-tetrahydro-5-hydroxy-2-methyl-4-pyrimidinecarboxylic acid are used.
